(19) United States
(12) Patent Application Publication
(10) Pub. No.: US 2006/0078581 A1
(43) Pub. Date: Apr. 13, 2006

(54) Oily gel composition

(75) Inventor: Naoya Yamato, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/281,601

(22) Filed: Nov. 18, 2005

Related U.S. Application Data
(63) Continuation of application No. PCT/JP04/05714, filed on Apr. 21, 2004.

(30) Foreign Application Priority Data
May 28, 2003 (JP) 2003-151576

(51) Int. Cl. A61K 8/92; A61K 8/42

(52) U.S. Cl. 424/401

(57) ABSTRACT

Oily gel compositions which contain at least one member of specific N-acyl-L-glutamic acid dialkylamides (component A), at least one member of polyhydric alcohol compounds (component B), and an oily substrate (component C), in which the ratio by weight of (component A)/(component B) is from 1/3 to 1/990, may be prepared by dissolving the gelling agent at a relatively low temperature and by using a rather small amount of a gelling agent. The oily gel composition is stable even at high temperatures, and does not afford a bad influence on touch or feeling when used on the skin or the hair.

### CROSS REFERENCES TO RELATED APPLICATIONS

This application is a continuation of International Patent Application No. PCT/JP2004/005714, filed on Apr. 21, 2004, and claims priority to Japanese Patent Application No. 2003-151576, filed on May 28, 2003, both of which are incorporated herein by reference in their entireties.

### BACKGROUND OF THE INVENTION

1. Field of the Invention

The present invention relates to oily gel compositions. More particularly, the present invention relates to oily gel compositions which are able to be prepared at a relatively low temperature, and which are relatively stable at high temperatures. The present invention further relates to oily gel compositions in which the compounding amount of a gelling agent is relatively small and for which, when used on the skin or the hair, no bad affection is noted in the feeling, etc. The present invention also relates to oily gel compositions which are able to be utilized as cosmetics or as aromatizers as well.

2. Discussion of the Background

There are known hydrophobic gels in which ethanol, ethylene glycol monoethyl ether, petroleum solvent, or natural hydrocarbon solvent is easily gelled by a higher fatty acid salt, particularly by sodium stearate, and they have been utilized as aromatizers (see, JP-B-56-6783 and JP-B-57-50502). However, in those hydrophobic gel aromatizers, a solvent such as water, glycol, or alcohol is an essential component for dissolving the sodium stearate which is used as the gel forming agent and, therefore, there is a disadvantage that such hydrophobic gel aromatizers are not resistant to heating.

Examples of other hydrophobic gel aromatizer agent are a solid gel aromatizer of hydrogenated castor oil with a hydrocarbon compound such as d-limonene and a solid gel aromatizer of 12-hydroxystearic acid with a hydrocarbon compound such as d-limonene. However, those hydrophobic gel aromatizers suffer from the disadvantage that they are far less resistant to heat as compared with the above-mentioned gel aromatizer of the sodium stearate type.

On the other hand, in JP-B-53-13434, it is disclosed that an N-acylamino acid amine salt or ester or amide derivative thereof has a gelling property for an oily substrate. In JP-B-3-80025, it is taught that an oily gel composition having good transparency may be prepared from the amide derivative as such and a volatile terpene hydrocarbon. Further, JP-A-2002-316971 discloses that N-2-ethylhexanoyl-L-glutamic acid dialkylamide has a high gelling property for an oily substrate. In JP-A-7-247475, it is disclosed that an aspartic acid phenylalanine cyclic dipeptide derivative has a high gelling property for an oily substrate. However, those methods are not satisfactory as manufacturing methods for oily gel compositions including cosmetics and aromatizers, because a relatively high temperature is required for dissolving the gelling agent, etc.

In JP-A-8-047635, an oily substrate is gelled using a fatty acid dextrin ester as a gelling agent, but any formulation thereof is compounded with 10 wt % or more of a gelling agent and, in addition, the gelling agent itself is a macromolecular compound having a relatively high molecular weight. Therefore, when the product is used for the skin or the hair, a coated feeling is strong, and that is not preferred.

In U.S. Pat. No. 5,650,144, a compounding example of 2% by weight of lauroylglutamic acid dibutylamide with 0.25% by weight of dipropylene glycol is disclosed (see, Example 18) and, in U.S. Pat. No. 5,958,386, a compounding example of 2% by weight of lauroylglutamic acid dibutylamide with 3.25% by weight of hexylene glycol is disclosed (see, Example 1) but, in the compounding amounts as such, the effect of lowering the dissolving temperature of lauroylglutamic acid dibutylamide is not sufficient.

Thus, there remains a need for oily gel compositions which do not suffer from the above-discussed drawbacks.

### SUMMARY OF THE INVENTION

Accordingly, it is one object of the present invention to provide novel oil gel compositions.

It is another object of the present invention to provide novel oily gel compositions in which the above-mentioned disadvantages are solved.

It is another object of the present invention to provide novel oily gel compositions in which a gelling agent is able to be dissolved at a relatively low temperature in preparing the oily gel composition.

It is another object of the present invention to provide novel oily gel compositions which are stable even at high temperatures.

It is another object of the present invention to provide novel oily gel compositions in which the compounding amount of a gelling agent is relatively small.

It is another object of the present invention to provide novel oily gel compositions which do not cause a bad feeling or touch when applied to the skin or the hair.

It is another object of the present invention to provide novel oily gel compositions which are homogeneous and which exist as a semi-transparent to turbid emulsion.

It is another object of the present invention to provide novel oily gel compositions which exhibit a good appearance.

It is another object of the present invention to provide novel oily gel compositions which are suitable for use in cosmetics or as aromatizers.

These and other objects, which will become apparent during the following detailed description, have been achieved by the inventor's discovery that an oily gel composition has a good property when it contains at least one member of specific N-acyl-L-glutamic acid dialkylamides (component A), at least one member of polyhydric alcohol compounds (component B) and an oily substrate (component C) such that the ratio by weight of (component A)/(component B) is from 1/3 to 1/990, that the N-acyl-L-glutamic acid dialkylamide is able to be dissolved at a relatively low temperature in preparing the above, that an oily gel composition which is stable even at high temperature is able to be prepared, that the gelling agent is able to be used in a relatively small amount, and that there is no bad influence to touch or feeling upon application to the skin or the hair. It has been also found that the resulting oily gel composition is a composition which is able to be utilized as a cosmetic or an aromatizer.

Thus, the present invention provides the following:

(1) An oily gel composition, comprising:

(A) at least one N-acyl-L-glutamic acid dialkylamide compound represented by formula (1): 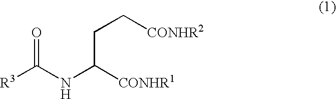
in which R¹ and R² each independently represent a C₁₋₂₆ hydrocarbon group and R³ represents a C₇₋₁₇ hydrocarbon group;

(B) at least one polyhydric alcohol compound, excluding glycerol; and

(C) at least one oily substrate,

wherein the ratio by weight of the at least one N-acyl-L-glutamic acid dialkylamide compound (A): the at least one polyhydric alcohol compound (B) is from 1:3 to 1:990.

(2) The oily gel composition according to the above (1), wherein, in the N-acyl-L-glutamic acid dialkylamide compound represented by the formula (1), R¹ and R² each independently represent a C₃₋₅ hydrocarbon group and R³ represents a C₇₋₁₇ hydrocarbon group.

(3) The oily gel composition according to the above (1) or (2), wherein, in the N-acyl-L-glutamic acid dialkylamide compound represented by the formula (1), R¹ and R² each independently represent a C₃₋₅ hydrocarbon group and R³ represents a C₇₋₉ hydrocarbon group.

(4) The oily gel composition according to any of the above (1) to (3), wherein the N-acyl-L-glutamic acid dialkylamide represented by the formula (1) is N-2-ethylhexanoyl-L-glutamic acid dibutylamide.

(5) The oily gel composition according to any of the above (1) to (4), wherein the at least one oily substrate (C) is at least one member selected from the group consisting of silicone oil, hydrocarbon oil, and mixtures thereof.

(6) The oily gel composition according to the above (5), wherein the at least one oily substrate (C) is one member selected from silicone oil.

(7) A cosmetic, which comprises an oily gel composition mentioned in any of the above (1) to (6).

(8) An aromatizer, which comprises an oily gel composition mentioned in any of the above (1) to (6).

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows an optical microscopic picture (×200) and a polarization microscopic picture (×200) of the gel prepared from N-lauroyl-L-glutamic acid dibutylamide, propylene glycol, and cyclomethicone (SH 245).

### DETAILED DESCRIPTION OF THE PREFERRED EMBODIMENTS

The present invention will now be illustrated in detail as hereunder.

The specific N-acyl-L-glutamic acid dialkylamide used in the present invention is represented by the following formula (1) and is a gelling agent for an oily substrate in the oily gel composition of the present invention. 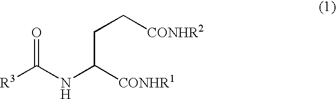
In the formula, R¹ and R² each independently represent a C₁₋₂₆ hydrocarbon group and R³ represents a C₇₋₁₇ hydrocarbon group.

In the oily gel composition of the present invention, at least one member of the N-acyl-N-glutamic acid dialkylamide compounds represented by the above formula (1) is compounded. In the N-acyl-L-glutamic acid dialkylamide as such, it is preferred that R¹ and R² each independently represent a C₃₋₅ hydrocarbon group and R³ represents a C₇₋₁₇ hydrocarbon group; more preferably, R¹ and R² each independently represent a C₃₋₅ hydrocarbon group and R³ represents a C₇₋₉ hydrocarbon group; and, particularly preferably, the N-acyl-N-glutamic acid dialkylamide compounds represented by the above formula (1) is N-2-ethylhexanoyl-L-glutamic acid dibutylamide.

The N-acyl-L-glutamic acid dialkylamide represented by the formula (1) may have one or more asymmetric carbon(s) depending upon the type(s) of R¹, R² and/or R³ and, in the gelling agent in the oily gel composition of the present invention, it is acceptable to use an optical isomer due to the asymmetric carbon as such, a stereoisomer such as diastereomer, a mixture of any of stereoisomers, or racemic substances. When R¹, R², and/or R³ have/has an olefinic double bond, its configuration may be any of Z and E and, in the oily gel composition of the present invention, a geometrical isomer in a pure state or a mixture of any of geometrical isomers may be used. Further, in the oily gel composition of the present invention, any of a hydrate or solvent of the N-acyl-L-glutamic acid dialkylamide compound represented by the above formula (1) may be used.

The N-acyl-L-glutamic acid dialkylamide compound represented by the above formula (1) may be appropriately produced by known methods. For example, a long-chain fatty acid halide can be reacted with L-glutamic acid in a basic catalyst by a Schotten-Baumann reaction, and the resulting N-acylated glutamic acid may be further subjected to a heating reaction with an amine derivative such as alkylamine in the presence of an acid catalyst or without catalyst to give a product.

In the meanwhile, with regard to the N-acyl-L-glutamic acid dialkylamide compound represented by the above formula (1), production examples of a part of the compound are illustrated in the Examples which will be mentioned later in a specific and detailed manner. Therefore, any of the N-acyl-L-glutamic acid dialkylamide compounds represented by the above formula (1) may be produced by anyone skilled in the art by an appropriate selection of starting material, reagent, reaction condition, etc. by referring to such production examples and, if necessary, by adding appropriate modification and variation to such process.

As to the component A for the oily gel composition of the present invention, one member of the above-mentioned N-acyl-L-glutamic acid dialkylamide may be used or a combination of two or more thereof may be used. With regard to the amount of the N-acyl-L-glutamic acid dialkylamide compound to be used in the oily gel composition of the present invention (compounding amount), there is no particular limitation so far as it is an amount which is able to make the oily substrate (component C) into a gel. Usually however, it is 0.1 to 20 part(s) by weight, preferably 0.5 to 10 part(s) by weight or, more preferably, 0.5 to 5 part(s) by weight per 100 parts by weight of the resulting oily gel composition. When the amount is less than 0.1 parts by weight, the resulting oily gel composition may sometimes not have sufficient gel strength while, while when it is more than 20 parts by weight, there are some cases in which dissolution is not achieved and in which the appearance of the resulting oily gel composition is deteriorated.

The role of the polyhydric alcohol which is used as a component B in the oily gel composition of the present invention is as a solubilizing agent for the component A. Examples of the polyhydric alcohol are ethylene glycol, diethylene glycol, triethylene glycol, propylene glycol, dipropylene glycol, polypropylene glycol, 1,3-butylene glycol, 3-methyl-1,3-butanediol, hexylene glycol, ethylhexanediol, polyethylene glycol, polyglycerol, sorbitol, maltitol, and mannitol and, in view of the property that the dissolving temperature of the N-acyl-L-glutamic acid dialkylamide is able to be lowered while an appropriate gelling ability is still maintained, preferred ones are ethylene glycol, diethylene glycol, propylene glycol, dipropylene glycol, and 1,3-butylene glycol. Incidentally, glycerol is not included therein because, in the preparation of an oily gel composition, a high temperature is needed for dissolving the N-acyl-L-glutamic acid dialkylamide which is a component A.

Although the amount of the polyhydric alcohol used (compounding amount) is not particularly limited, it is used, for example, in about 0.1 to 99 part(s) by weight, more preferably 5 to 90 part(s) by weight or, particularly preferably, 5 to 30 parts by weight per 100 parts by weight of the resulting oily gel composition. When the amount is less than 0.1 part by weight, there are some cases in which the dissolving temperature for the N-acyl-L-glutamic acid dialkylamide becomes high in the preparation of the oily gel composition while, when it is more than 99% by weight, there are some cases in which a sufficient gel strength is not available and, further, the compounding amount of a perfume becomes insufficient when used as an aromatizer while, and, when used as a cosmetic, a strong sticky feeling specific to polyhydric alcohol is noted upon use for the skin and the hair whereby that is not preferred.

The ratio of the N-acyl-L-glutamic acid dialkylamide compound which is the component A to the polyhydric alcohol which is the component B for the oily gel composition of the present invention, i.e., the ratio (component A)/(component B) (ratio by weight), is from 1/3 to 1/990, preferably from 1/5 to 1/99 and, more preferably, from 1/7 to 1/30. When the ratio of (component A)/(component B) is more than 1/3, the dissolving temperature of the N-acyl-L-glutamic acid dialkylamide may become high, it is unable to be dissolved well or the appearance of the resulting oily gel composition or the feeling upon its application to the skin or the hair may be deteriorated. When the ratio is less than 1/990, there may be the case in which the components B and C are separated or where a good gel is not formed.

With regard to the oily substrate which is the component C used for the manufacture of the oily gel composition of the present invention, there is no particular limitation so far as it is able to well dissolve the above gelling agent by heating and is able to form a gel upon cooling down to room temperature. Specific examples of the oily substrate are silicone oil such as methylphenylpolysiloxane, methylpolysiloxane, octamethyl cyclotetrasiloxane, a copolymer of dimethylsiloxane with methyl(polyoxyethylene) siloxane, decamethyl cyclopentasiloxane, a copolymer of dimethylsiloxane with methyl (polyoxyethylene-polyoxypropylene) siloxane, methyl hydrogen polysiloxane, dodecamethyl cyclohexasiloxane, methylpolycyclosiloxane, a copolymer of dimethylsiloxane with methylstearyloxy siloxane, methyl polysiloxane emulsion, octamethyl trisiloxane, highly-polymerized methyl polysiloxane, tetradecamethylhexasiloxane, trimethylsiloxysilicic acid, polyether-modified organopolysiloxane, fluoroalkyl polyoxyalkylene co-modified organopolysiloxane, alkyl-modified organopolysiloxane, terminal-modified organopolysiloxane, fluorine-modified organopolysiloxane, amodimethicone, cyclomethicone, amino-modified organopolysiloxane, silicone gel, acrylsilicone, and trimethylsiloxysilicic acid; higher alcohol such as cetyl alcohol, isostearyl alcohol, lauryl alcohol, hexadecyl alcohol, and octyldodecanol; fatty acids such as isostearic acid, undecylenic acid, and oleic acid; ester, such as myristyl myristate, hexyl laurate, decyl oleate, isopropyl myristate, hexyldecyl dimethyloctanoate, glycerol monostearate, diethyl phthalate, ethylene glycol monostearate, and octyl hydroxystearate; hydrocarbons such as d-limonene, α-pinene, liquid paraffin, Vaseline, and squalane; waxes such as lanoline, reduced lanoline, and carnauba wax; fats/oils such as mink oil, cacao oil, coconut oil, palm kernel oil, camellia oil, sesame oil, castor oil, and olive oil; etc.

The oily substrate is used preferably in about 1 to 99% by weight or, more preferably, in 10 to 90% by weight based on the total weight of the oily gel composition. When the compounding amount of the oily substrate is less than 1% by weight or more than 99% by weight, there may be the cases in which sufficient gel strength is not achieved or the dissolving temperature in the preparation of the oily gel composition becomes high. With regard to the oily substrate, it is also possible to use two or more oily substrates jointly.

There is no particular limitation for the manufacturing method of the oily gel composition of the present invention and, for example, a mixture of N-acyl-L-glutamic acid dialkylamide, a polyhydric alcohol compound, and an oily substrate may be heated at about 50 to 100° C. and then cooled down to room temperature with stirring or being allowed to stand whereupon the aimed oily gel composition is able to be prepared. With regard to the cooling with stirring and being allowed to stand, the former is usually preferred. That is because, especially when a silicone oil or hydrocarbon oil is used as the oily substrate, the polyhydric alcohol is sometimes not soluble in such an oily substrate and, in case acylglutamic acid dibutylamide is dissolved by heating and cooled thereafter without stirring, there are some cases in which the state of being separated into two layers (oily layer and polyhydric alcohol layer) continues and only a polyhydric alcohol layer is gelled. Alternatively, N-acyl-L-glutamic acid dialkylamide and a polyhydric alcohol compound are previously heated to dissolve and, when they give a homogeneous solution, an oily substrate is added thereto followed by cooling down to room temperature or, after N-acyl-L-glutamic acid dialkylamide and a polyhydric alcohol are heated to dissolve and the resulting mixed solution is added to an oily substrate to give the gel.

With regard to a polyhydric alcohol and an oily agent, they are not mixed each other sometimes depending upon each of their types and, in that case, when N-acyl-L-glutamic acid dialkylamide is heated to dissolve and then cooled down to room temperature with strong stirring in the preparation of an oily gel composition, a homogeneous gel is able to be prepared. The method for the cooling may be either by being allowed to stand at room temperature or by quick cooling using cold water. In the resulting oily gel composition, a product which is thought to be an associated product of a liquid crystal type may be sometimes observed.

In the oily gel composition of the present invention, another gelling agent in addition to the above-mentioned gelling agent may be used. Thus, a gelling agent used for an oily substrate such as a polyamide resin, 12-hydroxystearic acid, sodium stearate, aluminum octanoate, and dibenzylidene-D-sorbitol may also be used. It is also possible to use an aid for gelling such as 1,2-polybutadiene.

Further, water may be added to the oily gel composition of the present invention to give an oily gel of an emulsified type.

If necessary, the oily gel composition of the present invention may be compounded with a perfume, a surfactant, various additives, various powders, etc. There is no particular limitation for the type of such components but they may be appropriately selected depending upon the use of the oily gel composition and upon the desired property. Two or more of those components may be used jointly.

Examples of the perfume are natural plant perfumes such as rose oil, jasmine oil, neroli oil, and lavender oil; natural animal oils such as musk oil, civet oil, and castrium oil; synthetic hydrocarbons such as limonene and β-caryophyllene; synthetic alcohols such as cis-3-hexenol and linalool; synthetic aldehydes such as 2,6-nonadienal and citral; synthetic ketones such as β-ionone and cyclopentadecanone; synthetic esters such as linalyl acetate; synthetic lactones such as γ-undecalactone; synthetic phenols such as eugenol, synthetic oxides such as rose oxide; synthetic nitrogen-containing compounds such as indole; synthetic acetals such as phenylacetaldehyde dimethyl acetal; and synthetic Schiff bases such as aurantiol.

Examples of the surfactant are anionic surfactants such as N-long-chain acylamino acid salts (e.g., N-long-chain acylglutamates and N-long-chain acyl neutral amino acid salts), N-long-chain fatty acid acyl-N-methyltaurine salts, alkyl sulfates and alkylene oxide adducts thereof, fatty acid amide ether sulfates, fatty acid metal salts and weakly basic salt, surfactants of a sulfosuccinate type, alkyl phosphates and alkylene oxide adducts thereof, and alkyl ether carboxylic acids; nonionic surfactant such as surfactants of an ether type (e.g., glycerol ethers and alkylene oxide adducts thereof), surfactants of an ester type (e.g., glycerol esters and alkylene oxide adducts thereof), surfactants of an ether-ester type (e.g., sorbitan esters and alkylene oxide adducts thereof), surfactants of an ester type (e.g., polyoxyalkylene fatty acid esters, glycerol esters, fatty acid polyglycerol esters, sorbitan esters, and sucrose fatty acid ester), and nonionic surfactants of a nitrogen-containing type (e.g., alkyl glucosides, hydrogenated castor oil pyroglutamic acid diesters and ethylene oxide adducts thereof, and fatty acid alkanolamides); cationic surfactants such as aliphatic amines (e.g., alkylammonium chlorides and dialkylammonium chlorides) and quaternary ammonium salts thereof, aromatic quaternary ammonium salts (e.g., benzalkonium salts) and fatty acid acylarginine esters; and amphoteric surfactants (e.g., betaine-type surfactants such as carboxybetaine, aminocarboxylate-type surfactants, and imidazoline-type surfactants).

Examples of various additives are amino acid such as glycine, alanine, serine, threonine, arginine, glutamic acid, aspartic acid, leucine, and valine; glycerol; water-soluble macromolecular substance such as polyamino acid including polyglutamic acid, and polyaspartic acid as well as salt thereof, acacia, arginate, xanthan gum, hyaluronic acid, hyaluronate, chitin, chitosan, water-soluble chitin, carboxyvinyl polymer, carboxymethylcellulose, hydroxyethylcellulose, hydroxypropyl trimethylammonium chloride, poly dimethylmethylenepiperidium chloride, polyvinylpyrrolidone derivative quaternary ammonium, cationized protein, decomposed collagen and derivative thereof, acylated protein and polyglycerol; adducts of a sugar alcohol with alkylene oxide; as well as extract of plants and animals, nucleic acids, vitamins, enzymes, anti-inflammatory agents, bactericides, antiseptic agents, antioxidants, ultraviolet absorbers, chelating agents, antiperspirants, pigments, dyes, oxidation dyes, organic and inorganic powders, pH-adjusting agents, pearling agents, and moisturizers.

Examples of various powder are powders of resin such as Nylon beads, Nylon powder, and silicone beads; inorganic powders such as metal fatty acid soaps, yellow iron oxide, red iron oxide, black iron oxide, chromium oxide, cobalt oxide, carbon black, ultramarine, Prussian blue, zinc oxide, titanium oxide, zirconium oxide, silicon oxide, aluminum oxide, cerium oxide, mica titanium, boron nitride, barium sulfate, calcium carbonate, magnesium carbonate, aluminum silicate, magnesium silicate, silicon carbide, dye, lake, sericite, mica, talc, kaolin, plate-shaped barium sulfate, butterfly-shaped barium sulfate, fine particles of titanium oxide, fine particles of zinc oxides, and fine particle of iron oxide; and acylamino acids such as acyllysine, acylglutamic acid, and acylglycine. Such powder may be subjected to a surface treatment such as treatment with silicone, treatment with a fluorine compound, treatment with a silane coupling agent, treatment with a silane and with an organic titanate, treatment with acylated lysine, treatment with a fatty acid, treatment with a metal soap, treatment with oil, and treatment with an amino acid.

There is no particular limitation for the use of the oily gel composition of the present invention, and the composition may be used for cosmetic/perfumery such as aromatizers and cosmetics. For example, it may be used as an aromatizer such as that for automobiles and for domestic use, as a cosmetic for gel cosmetic, packed cosmetic, and granular cosmetic and as a candle. Depending upon the object, it is able to be prepared as a composition in an appropriate shape by a conventional method in an appropriate manner except that the oily gel composition of the present invention is compounded therewith and contained therein.

Other features of the invention will become apparent in the course of the following descriptions of exemplary embodiments which are given for illustration of the invention and are not intended to be limiting thereof.

### EXAMPLES

In the following examples, the lauroylglutamic acid dibutylamide is a gelling agent of an amino acid type “GP-1” which is manufactured by Ajinomoto.

### Manufacturing Example 1

### Manufacture of N-2-ethylhexanoyl-L-glutamic acid dibutylamide (Part 1)

Sodium L-glutamate monohydrate (110 g) was dissolved in a mixed solution of 140 g of water and 78 g of a 27% aqueous solution of sodium hydroxide, and the solution was cooled down to 10° C. Acetone (110 g) was added thereto, and then 87 g of 2-ethylhexanoyl chloride and 78 g of a 27% aqueous solution of sodium hydroxide were dropped thereinto. The resulting acylated reaction solution was diluted with 100 g of water and neutralized with 63 g of 95% sulfuric acid to separate into oil and water. The aqueous layer was removed, and the oily layer was concentrated in vacuo to give an oily substance. The oily substance was dissolved in 742 g of methanol, 6.2 g of 95% sulfuric acid was added thereto, and the mixture was heated to reflux for 9 hours. The reaction solution was allowed to cool down to 35° C. and neutralized with 8.8 g of n-butylamine, and methanol was evaporated therefrom to give an oily substance again. Toluene (643 g) and 271 g of n-butylamine were added to the oily substance, and the mixture was heated at 90° C. for 10 hours with stirring. To this were added 506 g of hot water and 130 g of 95% sulfuric acid to separate into oil and water and the aqueous layer was removed. To the oily layer was added 1,200 g of hot water, and the solvent was evaporated at ordinary pressure to give a slurry of white solid. The solid was filtered and dried in vacuo to give 2-ethylhexanoyl-L-glutamic acid dibutylamide.

(a) ¹³C-NMR peaks (solvent: CDCl₃): 12.04, 12.07, 13.74, 13.96, 13.99, 20.08, 20.11, 22.70, 22.74, 26.01, 29.83, 31.56, 31.60, 32.37, 33.05, 39.29, 39.53, 49.37, 52.53, 52.56, 171.29, 173.03, 176.66 (ppm).

(b) ¹H-NMR peaks (CDCl₃): δ: 3.248 (m, 4H), 4.373 (m, 1H), 6.199 (brs, 1H), 7.079 (brs, 1H), 7.169 (brs, 1H).

(c) wave numbers of infrared absorption spectrum: 3291.7, 2961.0, 2932.5, 1638.2, 1551.2, 1452.6 (cm⁻¹).

(d) MS spectrum: 382.3 (M-H)⁻.

### Manufacturing Example 2

### Manufacture of N-2-ethylhexanoyl-L-glutamic acid dibutylamide (Part 2)

Sodium L-glutamate monohydrate (57.6 g) was dissolved in a mixed solution of 92.6 of water, 72.9 g of IPA, and 41 g of a 27% aqueous solution of sodium hydroxide, and the mixture was cooled down to 10° C. 2-Ethylhexanoyl chloride (50.1 g) and 49.6 g of a 27% aqueous solution of sodium hydroxide were simultaneously dropped into the solution during 1.5 hours while keeping the pH at 11 (±0.2) and the temperature at 10 (±5° C.) and, after that, the mixture was raised to 30° C. followed by stirring for 1 hour. The resulting acylated solution was neutralized with 41.2 g of 75% sulfuric acid where the temperature was kept at 40° C. or lower to adjust to pH 1.9, whereupon separation into oil and water took place. The aqueous layer was removed, and the oily layer was concentrated in vacuo (at 50° C., in vacuo) to give an oily substance. To the oily substance were added 151.9 g of water, 91.3 g of n-butanol, and 496.1 g of toluene, and the mixture was separated into oil and water at 40° C. To the resulting oil layer were added 21.4 g of boron oxide and 61.9 g of butylamine, the mixture was refluxed using an oil bath (bath temperature: 135° C.), and azeotropic dehydration of the resulting water was carried out during 13 hours. To this was added 444 g of diluted (concentration: about 6%) sulfuric acid to separate into oil and water at 85° C. To the obtained oily layer was added 419 g of water to separate into oil and water once again. The resulting oily layer was subjected to an azeotropic treatment in vacuo together with gradual dropping of 1,000 g of water thereinto to remove n-butanol and toluene, whereupon an aqueous slurry of a white solid was prepared. The white solid was filtered and dried in vacuo to give 105 g of N-2-ethylhexanoyl-L-glutamic acid dibutylamide (yield: 89%).

### Comparative Examples 1 to 4 and Examples 1 to 18

### Test of the Oily Gel Composition

Compositions comprising components A to C as described in Tables 1 to 3 below were tested as follows.

(1) About 5 g of each composition was placed in a 10-ml vial and heated up to 100° C. to check whether the component A was completely dissolved (using the following Evaluation Method 1).

(2) Both samples in which the component A was completely dissolved and those in which the component A was not dissolved (including the case where it was partially dissolved) were cooled down to room temperature, and the dissolved state (whether gelled or not) of the sample cooled down to room temperature was checked by naked eye (using the following Evaluation Method 2).

(3) The sample which was gelled when cooled down to room temperature in (2) was stored for one week in a constant-temperature bath of 40° C., and the dissolved state at that time was checked by naked eye (using the following Evaluation Method 3).

(4) The sample which was gelled when cooled down to room temperature in (2) which was an oily gel was applied to the skin and the smoothness was evaluated (using the following Evaluation Method 4).

### Evaluation Method 1

### Dissolving of N-acyl-L-glutamic acid dialkylamide at 100° C.

When gel compositions in various formulations were heated at 100° C., the case in which N-acyl-L-glutamic acid dialkylamide was completely dissolved was evaluated as “o” while the case in which it was not dissolved was evaluated as “x”.

### Evaluation Method 2

### Gelling

When gel compositions in various formulations were heated at 100° C. followed by being allowed to stand to cool down to room temperature, and the dissolved state was checked by naked eye, the case in which it was completely gelled was evaluated as “o” while the case in which it was not gelled was evaluated as “x”.

### Evaluation Method 3

### Stability of the Oily Gel at High Temperature

When the product which was gelled in the above Evaluation method 2 was kept at 40° C. for one week, the case in which the oily gel was completely gelled was evaluated as “o,” the case in which it was partially separated was evaluated as “Δ,” and the case in which it was liquefied was evaluated as “x”.

### Evaluation Method 4

### Sensory Test Upon Application to the Skin (Smoothness)

When the product which was completely gelled in the above Evaluation method 2 was applied to the skin, the resulting feeling (smoothness) was subjected to the following five-stage evaluation by professional panelists:

5: very smooth

4: smooth

3: normal

2: a bit sticky

1: sticky

When an average mark was 3.5 or more, it was evaluated as “OO;” when it was from 3.0 or more to less than 3.5, it was evaluated as “O;” when it was from 2.5 or more to less than 3.0, it was evaluated as “Δ;” and, when it was less than 2.5, it was evaluated as “x”.

TABLE 1

| | | | | | | | | |
|---|---|---|---|---|---|---|---|---|
| CE 1 | CE 2 | CE 3 | Ex 1 | Ex 2 | Ex 3 | Ex 4 | Ex 5 | Ex 6 |

Component A

| | | | | | | | | | |
|---|---|---|---|---|---|---|---|---|---|
| N-2-Ethylhexanoylglutamic acid | 2 | 1 | 2 | 2 | 2 | 2 | 1 | 1 | 1 |
| dibutylamide | | | | | | | | | |
| N-Lauroylglutamic acid | | | | | | | | | |
| dibutylamide | | | | | | | | | |

Component B

| | | | | | | | |
|---|---|---|---|---|---|---|---|
| 1,3-BG | 1 | 15 | 20 | 30 | 10 | 15 | 20 |
| Propylene glycol | | | | | | | |
| Dipropylene glycol | | | | | | | |

Component C

| | | | | | | | | | |
|---|---|---|---|---|---|---|---|---|---|
| Cyclomethicone | 98 | | 97 | 83 | 78 | 68 | | | |
| Squalane | | 99 | | | | | 89 | 84 | 79 |

Test Items

| | | | | | | | | | |
|---|---|---|---|---|---|---|---|---|---|
| Dissolving at 100° C. | X | X | X | ο | ο | ο | ο | ο | ο |
| Gelling | X | Δ | X | ◯ | ◯ | ◯ | ◯ | ◯ | ◯ |
| Stability at High Temp | | | | ◯ | ◯ | ◯ | ◯ | ◯ | ◯ |
| Smoothness | | | | ◯◯ | ◯◯ | ◯ | ◯◯ | ◯◯ | ◯ |

Unit: % by weight
Ex: Example;
CE: Comparative Example

TABLE 2

| | | | | | | | | |
|---|---|---|---|---|---|---|---|---|
| CE 4 | Ex 7 | Ex 8 | Ex 9 | Ex 10 | Ex 11 | Ex 12 | Ex 13 | Ex 14 |

Component A

| | | | | | | | | |
|---|---|---|---|---|---|---|---|---|
| N-2-Ethylhexanoylglutamic acid | | | | | | | 4 | |
| dibutylamide | | | | | | | | |
| N-Lauroylglutamic acid | 2 | 2 | 2 | 2 | 2 | 4 | 2 | 2 |
| dibutylamide | | | | | | | | |

Component B

| | | | | | | | | |
|---|---|---|---|---|---|---|---|---|
| 1,3-BG | | | | | | | | |
| Propylene glycol | 10 | 15 | 20 | 30 | 50 | 30 | 50 | 70 |
| Dipropylene glycol | | | | | | | | |

Component C

| | | | | | | | | | |
|---|---|---|---|---|---|---|---|---|---|
| Cyclomethicone | 98 | 88 | 83 | 78 | 68 | 46 | 66 | 48 | 28 |
| Squalane | | | | | | | | | |

Test Items

| | | | | | | | | | |
|---|---|---|---|---|---|---|---|---|---|
| Dissolving at 100° C. | X | ◯ | ◯ | ◯ | ◯ | ◯ | ◯ | ◯ | ◯ |
| Gelling | X | ◯ | ◯ | ◯ | ◯ | ◯ | ◯ | ◯ | ◯ |
| Stability at High Temp | | Δ | ◯ | ◯ | Δ | ◯ | ◯ | Δ | Δ |
| Smoothness | | ◯◯ | ◯◯ | ◯◯ | ◯ | ◯ | ◯ | ◯ | ◯ |

Unit: % by weight
Ex: Example;
CE: Comparative Example

TABLE 3

| | Ex 15 | Ex 16 | Ex 17 | Ex 18 |
|---|---|---|---|---|
| Component A | | | | |
| N-2-Ethylhexanoylglutamic acid | 2 | 2 | 2 | 2 |
| dibutylamide | | | | |
| N-Lauroylglutamic acid dibutylamide | | | | |
| Component B | | | | |
| 1,3-BG | | | | |
| Propylene glycol | | | | |
| Dipropylene glycol | 20 | 30 | 15 | 20 |
| Component C | | | | |
| Cyclomethicone | 78 | 68 | | |
| Squalane | | | 83 | 78 |
| Test Items | | | | |
| Dissolving at 100° C. | ◯ | ◯ | ◯ | ◯ |
| Gelling | ◯ | ◯ | ◯ | ◯ |
| Stability at High Temp | ◯ | ◯ | ◯ | ◯ |
| Smoothness | ◯◯ | ◯ | ◯◯ | ◯◯ |

Unit: % by weight
Ex: Example;
CE: Comparative Example

### Examples 19 to 20

### Foundation

With regard to the foundations having the formulations as shown in the following Table 4, N-acyl-L-glutamic acid dibutylamide was able to be completely dissolved even at 100° C. or lower in such formulations and, in addition, the resulting foundation showed good stability and smoothness and provided a good feeling as well.

TABLE 4
Foundation

| | Example 19 | Example 20 |
|---|---|---|
| Talc | 43.1 | 43.1 |
| Kaolin | 15 | 15 |
| Sericite | 10 | 10 |
| Zinc white | 7 | 7 |
| Titanium dioxide | 3.8 | 3.8 |
| Red iron oxide | 1 | 1 |
| Yellow iron oxide | 2.9 | 2.9 |
| Black iron oxide | 0.2 | 0.2 |
| Squalane | 8.7 | 8.7 |
| POE Sorbitan monooleate | 3 | 3 |
| Isocetyl octanoate | 2 | 2 |
| N-2-Ethylhexanoylglutamic acid | 0.3 | |
| dibutylamide | | |
| N-Lauroylglutamic acid dibutylamide | | 0.3 |
| 1,3-BG | 3 | |
| Dipropylene glycol | | 3 |

Unit: % by weight

### Examples 21 to 22

### Makeup Remover in Gel

With regard to the makeup removers in gel having the formulations as shown in the following Table 5, N-acyl-L-glutamic acid dibutylamide was able to be completely dissolved even at 100° C. or lower in such formulations and, in addition, the resulting makeup remover showed good stability and smoothness and provided a good feeling as well and, further, it has a good fundamental property as a makeup remover.

TABLE 5
Makeup Removers in Gel

| | Example 21 | Example 22 |
|---|---|---|
| Liquid paraffine | 40 | 40 |
| POE (15) isostearate | 7 | 7 |
| Propylene glycol | 50 | 50 |
| N-2-Ethylhexanoylglutamic acid | 3 | |
| dibutylamide | | |
| N-Lauroylglutamic acid dibutylamide | | 3 |

Unit: % by weight

### Examples 23 to 24

### Cleansing Oil in Gel (Part 1)

With regard to the cleansing oils in gels having the formulations as shown in the following Table 6, N-acyl-L-glutamic acid dibutylamide was able to be completely dissolved even at 100° C. or lower in such formulations and, in addition, the resulting cleansing oil showed good stability and smoothness and provided a good feeling as well and, further, it has a good fundamental property as a cleansing oil.

TABLE 6
Cleansing Oil in Gel

| | Example 23 | Example 24 |
|---|---|---|
| Liquid paraffin | 40 | 40 |
| 2-Ethylhexyl stearate | 10 | 10 |
| Silicone oil | 25 | 25 |
| POE Oleyl alcohol ether | 7 | 7 |
| Dipropylene glycol | 15 | 15 |
| N-2-Ethylhexanoylglutamic acid | 3 | |
| dibutylamide | | |
| N-Lauroylglutamic acid dibutylamide | | 3 |

Unit: % by weight

### Examples 25 to 26

### Cleansing Oil in Gel (Part 2)

With regard to the cleansing oils in gels having the formulations as shown in the following Table 7, N-acyl-L-glutamic acid dibutylamide was able to be completely dissolved even at 100° C. or lower in such formulations and, in addition, the resulting cleansing oil showed good stability and smoothness, provided a good feeling as well, and, further, it has a good fundamental property as a cleansing oil.

TABLE 7
Cleansing Oil in Gel

| | Example 25 | Example 26 |
|---|---|---|
| Titanium oxide | 4.5 | 4.5 |
| Red No. 201 | 0.5 | 0.5 |
| Red No. 202 | 2 | 2 |
| Red No. 223 | 0.05 | 0.05 |
| Ceresin | 4 | 4 |
| Candelila wax | 4 | 4 |
| Carnauba wax | 2 | 2 |
| Castor oil | 30 | 30 |
| Isostearic acid diglyceride | 30.95 | 30.95 |
| POE (25) POP (20) 2-tetradecyl ether | 1 | 1 |
| Propylene glycol | 10 | 10 |
| N-2-Ethylhexanoylglutamic acid | 2 | |
| dibutylamide | | |
| N-Lauroylglutamic acid dibutylamide | | 2 |

Unit: % by weight

Microscopic Pictures of the Oily Gel Composition.

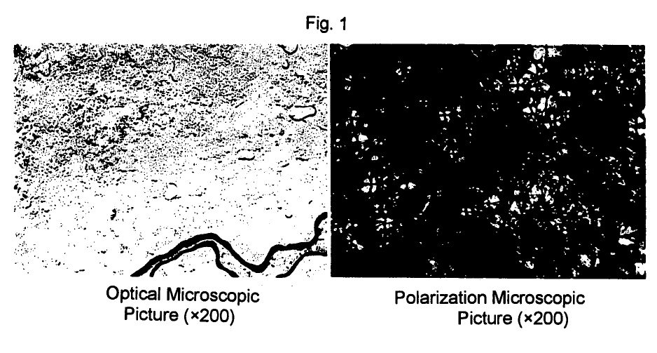

N-Lauroyl-L-glutamic acid dibutylamide (0.2 g), 2.0 g of propylene glycol, and 7.8 g of cyclomethicone (SH 245) were placed in a 50-ml glass vial bottle and homogeneously dissolved at 100° C. It was cooled by strongly stirring with hand to give an oily composition in gel. As shown in FIG. 1, the resulting gel shows no clear crystal under an optical microscope while it shows star-like stripes (crosses) under a polarization microscope and that strongly suggests the presence of an associate having a liquid crystal structure.

### INDUSTRIAL APPLICABILITY

In accordance with the present invention, there is easily provided oily gel compositions which are able to be prepared at a relatively low temperature, and which are stable even in a state of high temperature. Moreover, the compounding amount of the gelling agent is relatively small and, when applied to the skin or the hair, no bad effect is given to the feeling, etc.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An oily gel composition, comprising:
(A) at least one N-acyl-L-glutamic acid dialkylamide compound represented by formula (1): 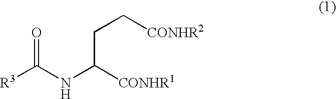
wherein R¹ and R² each independently represent a C₁₋₂₆ hydrocarbon group and R³ represents a C₇₋₁₇ hydrocarbon group;
(B) at least one polyhydric alcohol, excluding glycerol; and
(C) at least one oily substrate,
wherein the ratio by weight of said at least one N-acyl-L-glutamic acid dialkylamide compound (A): said at least one polyhydric alcohol (B) is from 1:3 to 1:990.

2. The oily gel composition according to claim 1, wherein, in formula (1), R¹ and R² each independently represent a C₃₋₅ hydrocarbon group and R³ represents a C₇₋₁₇ hydrocarbon group.

3. The oily gel composition according to claim 1, wherein, in formula (1), R¹ and R² each independently represent a C₃₋₅ hydrocarbon group and R³ represents a C₇₋₉ hydrocarbon group.

4. The oily gel composition according to claim 1, wherein said at least one N-acyl-L-glutamic acid dialkylamide compound represented by the formula (1) comprises N-2-ethylhexanoyl-L-glutamic acid dibutylamide.

5. The oily gel composition according to claim 1, wherein said at least one oily substrate (C) comprises at least one member selected from the group consisting of a silicone oil, a hydrocarbon oil, and mixtures thereof.

6. The oily gel composition according to claim 2, wherein said at least one oily substrate (C) comprises at least one member selected from the group consisting of a silicone oil, a hydrocarbon oil, and mixtures thereof.

7. The oily gel composition according to claim 3, wherein said at least one oily substrate (C) comprises at least one member selected from the group consisting of a silicone oil, a hydrocarbon oil, and mixtures thereof.

8. The oily gel composition according to claim 4, wherein said at least one oily substrate (C) comprises at least one member selected from the group consisting of a silicone oil, a hydrocarbon oil, and mixtures thereof.

9. The oily gel composition according to claim 1, wherein said at least one oily substrate (C) comprises a silicone oil.

10. The oily gel composition according to claim 2, wherein said at least one oily substrate (C) comprises a silicone oil.

11. The oily gel composition according to claim 3, wherein said at least one oily substrate (C) comprises a silicone oil.

12. The oily gel composition according to claim 4, wherein said at least one oily substrate (C) comprises a silicone oil.

13. A cosmetic composition, which comprises an oily gel composition according to claim 1.

14. A cosmetic composition, which comprises an oily gel composition according to claim 2.

15. A cosmetic composition, which comprises an oily gel composition according to claim 3.

16. A cosmetic composition, which comprises an oily gel composition according to claim 4.

17. An aromatizer, which comprises an oily gel composition according to claim 1.

18. An aromatizer, which comprises an oily gel composition according to claim 2.

19. An aromatizer, which comprises an oily gel composition according to claim 3.

20. An aromatizer, which comprises an oily gel composition according to claim 4.
